(19) United States
(12) Patent Application Publication
(10) Pub. No.: US 2012/0165611 A1
(43) Pub. Date: Jun. 28, 2012

(54) LAPAROSCOPIC ACCESS PORT AND PORT SLEEVE ARRANGEMENT

(76) Inventors: Neil Warren, Cardiff (GB); Andrea Potter, Windsor (GB); Robin Crossley, Windsor (GB); Jeremy Gardner, Windsor (GB); Ian Dera, Windsor (GB)

(21) Appl. No.: 13/322,652

(22) PCT Filed: May 27, 2010
(86) PCT No.: PCT/GB2010/050884

§ 371 Date: Jan. 30, 2012

(30) Foreign Application Priority Data
May 29, 2009 (GB) 0909393.1
Oct. 22, 2009 (GB) 0918565.3

(51) Int. Cl. A61B 1/32

(52) U.S. Cl. 600/204; 600/206

(57) ABSTRACT

Disclosed are embodiments of a laparoscopic access port having a head (portion 450) for use generally externally of a patient and for providing a seal around a laparoscopic tool (not shown)when said tool is inserted into the head via a (bore 408); and a sleeve portion (410) connected to the head and for accommodating the tool, the sleeve (410) extending along an axis and being adapted for inserting generally into the body of a patient in the direction of the axis;said access port being adjustable in overall length X in the direction of the axis, the sleeve portion comprising a shank (411) attached or attachable to the head (450) and a flanged piece (413) moveable relative to the shank by means of a mechanism for causing the overall length X of the port to adjust, the mechanism being operable externally of the patient at or adjacent the head, for example by means of rotation of an element of a lower head part (440), or by sliding motion of the shank (411) and flanged piece (413).

The invention relates to an access port and port sleeve arrangement for use in laparoscopic surgical procedures and the like. Laparoscopic surgery, often termed ‘key-hole’ surgery conventionally involves making, typically two or more, small incisions in a patient and inserting an access port into each of those incisions. One or more of the known access ports may allow the introduction of an insufflation gas so that a surgeon has room to undertake a procedure, for example by inflating the abdominal cavity of a patient. The access port further includes an opening through which is inserted a surgical tool during the procedure. Such apertures are generally self-sealing so when a tool is not present in the aperture, little insufflation gas pressure is lost through the port.

The known access ports have a stem which is inserted into a patient, for carrying a head of the access port typically having a self sealing aperture and insufflation gas valves etc. The stems need to be long enough to reach through bodily tissues of large or obese patients and so these stems are usually and needlessly too long for many patients. In addition, the stems are usually pointed so that they can be inserted easily into an incision. As a result of their length and pointed end of the stem, there is a possibility that internal trauma may occur if the stem is forced against internal tissues.

The inventor has realised that there is a need for an access port which avoids or mitigates internal trauma.

Commercially available access ports are, by necessity cumbersome and bulky. The ports usually have a means for insufflating the patient, as well as a self-sealing aperture. The ports are designed for use during surgery, and then removal when surgery is finished. Some surgical procedures require two or more sessions spaced hours or days apart. When an access port is reintroduced into a patient for further surgical procedures, for example, to check on the initial surgery, that reintroduction can cause additional trauma at the re-entry area.

The inventor has realised also that there is a need for a port which can be left in the patient for further surgical sessions, but the present devices mentioned above are not suitable for that purpose. A particular shortcoming of present designs is that they protrude into the body in use and protrude above the line of the body significantly, making them potentially very uncomfortable for the patient if the patient were to lie on the port or knock the head of the port on an obstruction. The necessary rigidity of the port also makes them dangerous if they were left in place.

The inventor has further realised that a device that can facilitate removal and reinsertion of an access port, and which could be left in place until all surgical sessions were complete, would be of great benefit to a patient and surgical staff.

Embodiments of the invention address the problems mentioned above. The access port of the present invention, in embodiments, employs a length adjustable stem, for example a telescopic stem principle, so that the conventional stem of the port is replaced by a length adjustable sleeve arrangement and this sleeve arrangement allows the internal length of the port (conventionally the stem portion) to be adjusted to the dimension of the patient's bodily tissues, for example the abdominal wall, so there is less chance of accidental internal trauma. The sleeve can be at least partially removeable from the head of the port so that the sleeve can be left in the patient if required, for example either to be reconnected later or to allow access to the surgical site for other instruments inserted through the sleeve. A removable cap can be used to seal the body cavity from the external environment. In addition, the same length adjustable principle can be applied to a stand-alone sleeve which is positioned generally inside the body without the head of an access port, which sleeve has little material protruding above the patient's body line and so can remain in the patient after an initial surgical procedure session, providing a ready reinsertion point, for example, for known access ports.

The present invention provides a laparoscopic access port having a head portion for use generally externally of a patient, and a sleeve portion extending along an axis and for inserting generally into the body of a patient in the direction of the axis, said port being adjustable in overall length in the direction of the axis, the sleeve portion comprising a shank attached or attachable to the head and a flanged piece moveable relative to the shank by means of a mechanism for causing the overall length of the port to adjust, the mechanism being operable externally of the patient at or adjacent the head.

The head, or part of the head, and sleeve may be separable.

The invention provides also a sleeve insertable at least partially into a patient, the sleeve being suitable for either complementary connection to the head of an access port or for removably supporting the head of a laparoscopic access port in use.

In an embodiment, said sleeve (in its stand-alone form or the sleeve attached or attachable to the head of an access port) includes a first seal member for preventing or inhibiting the escape of insufflation gases, in use, and a second seal member for preventing or inhibiting the escape of said gases, acting between the sleeve and the patient in the form of a flange or flanged piece.

In an embodiment, said sleeve is generally tubular, including a first tubular piece forming the shank and a second tubular piece forming the flanged piece, one of said tubular piece being relatively moveable within the other to adjust the overall length of the port.

Preferably the first or second tubular piece has a thread and the other of the first or second tubular pieces has a complementary thread or thread- following formation such that the movement of the second tubular piece causes said overall length adjustment.

Preferably the rotation of the second tubular piece is caused by a manually rotatable third tubular piece forming part of the mechanism and extending from the head such that the third tubular piece can be rotated externally of the patient in use.

Preferably said third tubular piece extends at least partially within the second tubular piece, which in turn is at least partially within the first tubular piece.

Preferably the third tubular piece includes at least one axially extending slot for accepting a detent formed on or in the second tubular piece for allowing the detent of second tubular piece to move axially in the slot under the influence of the thread.

Preferably, the flanged piece includes a distal end at which is formed a flange, optionally being formed from flexible material.

Preferably the sleeve further includes a one-way movement mechanism operable so that the shank and flanged piece can move only toward each other, in turn so that the flange of the flanged piece and the head can only move toward each other.

More preferably the one-way mechanism includes complementary formations on the shank and flanged piece which allow them only to come closer together.

In an embodiment the shank and flanged tubular piece of the sleeve include cooperating means to prevent or restrain their relative movement, for example during insertion of the sleeve.

Preferably said means to prevent or restrain relative movement includes a bayonet type mechanism.

Preferably the bayonet type mechanism includes one or more studs on one of the shank or flanged piece slideable in a complementary channel or channels in the other of the shank or flanged piece, the channel or channels including an area which allows the relative sliding of the shank and flanged piece and an area that does not.

Preferably the area of the channel or channels which allows said sliding together has sides which extend generally parallel to the axis and the area that does not has sides which extend obliquely to the axis.

In an embodiment, the flanged piece has at least one flexible draw tab for holding the flange in place while the shank is moved toward or over the flanged piece.

Preferably the draw tab comprises two draw tabs, which are tensioned in use to pull the shank or flanged piece together.

Preferably each draw tab is held in place in use to thereby hold the inner and outer flanges in place relative to each other.

The port may further include a plug or cap to substantially seal the port after the head of the access port is removed or partially removed.

In an embodiment, the access port further includes an introduction tool which includes a shoulder, in use engageable with the second tubular piece.

Preferably the access port includes at least one ear at or adjacent the head for allowing securing of the head by means of suture.

The invention extends to a combination of a sleeve for insertion into a patient optionally with features mentioned above, supporting a laparoscopic access port head and arranged such that the access port head is removable from the sleeve. The sleeve, if a stand-alone sleeve, can allow multiple reinsertions of the head onto the sleeve, or if the sleeve is attachable to the head of a port then the sleeve allows reconnection of the head at a later time while the sleeve stays in the patient.

The invention further extends to a method of using medical equipment, comprising or including the following steps in any suitable order:

a) inserting a sleeve into a patient for holding or supporting a laparoscopic access port head,

b) inserting a laparoscopic access port head into the sleeve or attaching a laparoscopic access port head to the sleeve;

c) optionally inserting and removing a surgical tool into the access port;

d) removing the laparoscopic access port or port head from the patient but not the sleeve;

e) optionally, reintroducing the laparoscopic access port into the port or re-attaching a laparoscopic access port head to the sleeve; and

f) removing at least a portion of the sleeve.

Preferably, the step of inserting the sleeve is followed by securing the sleeve in place, by means the bringing together of inner and outer flanges of the sleeve.

Preferably the sleeve may be sealed with out without the inserted or attached head, by means of the sealing cap fitted to an exterior portion of the sleeve, or fitted to the head.

Although, there are many ways of putting the invention into effect, by way of example only, embodiments of the invention are described below, with reference to the drawings, wherein:

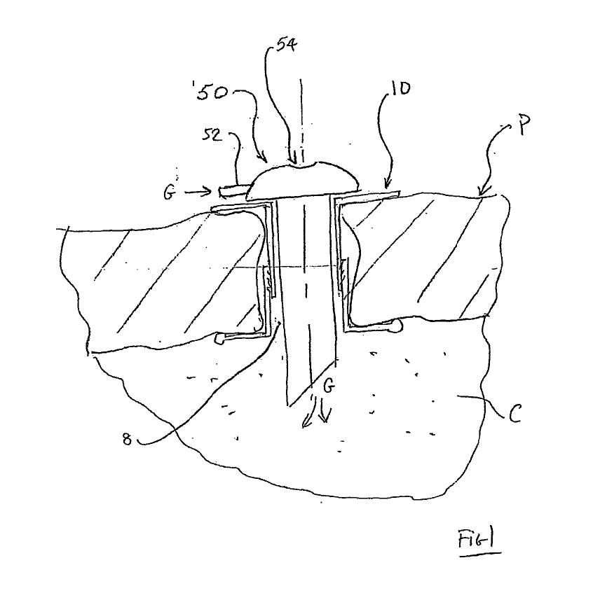

FIG. 1 shows the a first embodiment of invention in use;

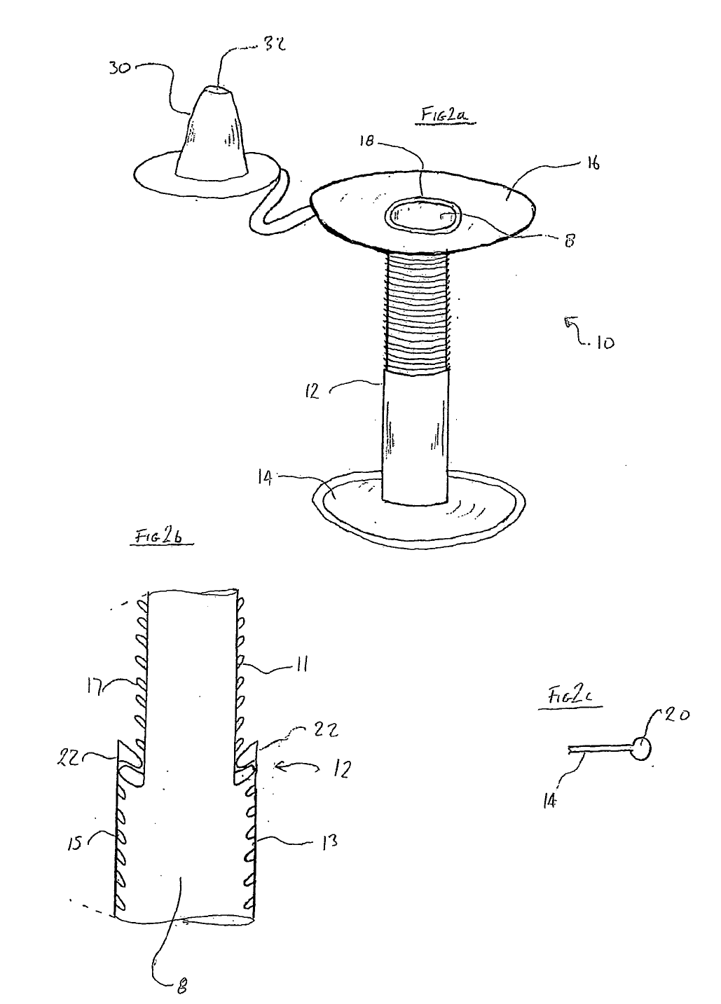

FIG. 2a shows a pictorial view the first embodiment of the invention;

FIGS. 2b and 2c show partial enlarged views of the first embodiment shown in FIG. 2a;

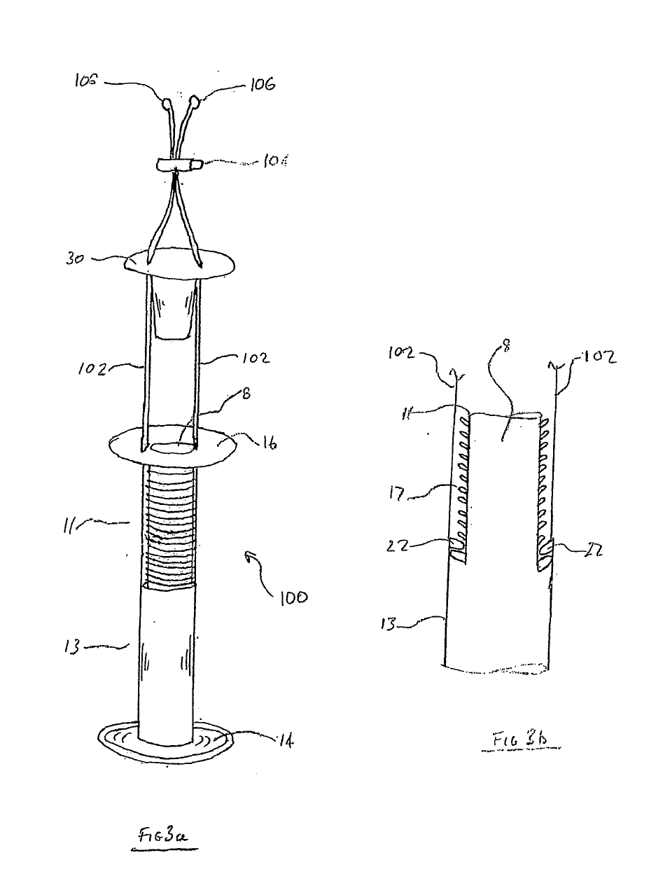

FIG. 3a shows a pictorial view of a second embodiment of the invention;

FIG. 3b shows a partial enlarged view of the second embodiment;

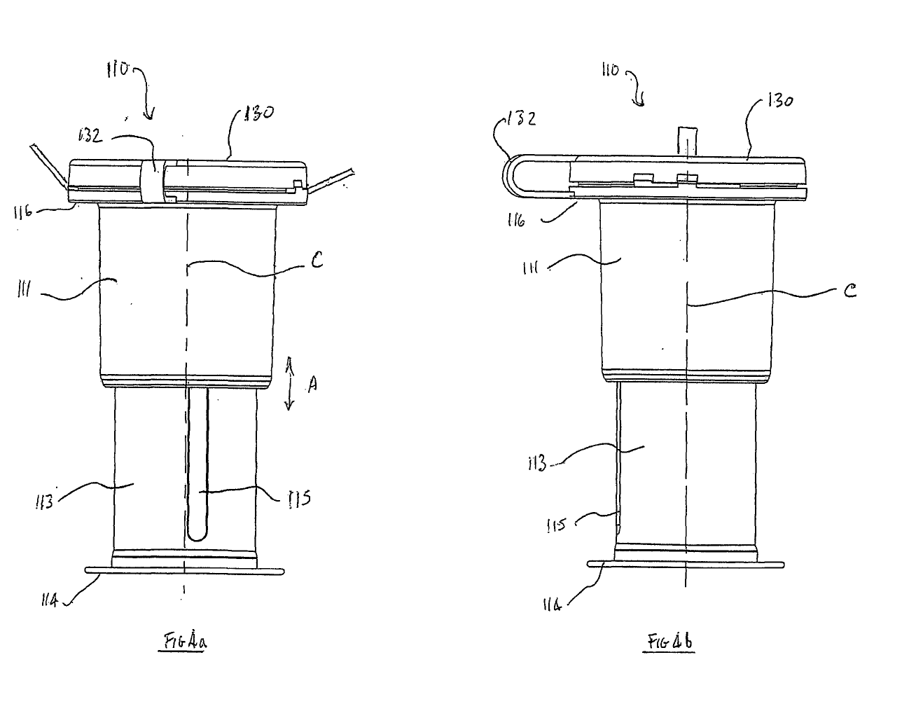

FIGS. 4a and 4b show side views of a third embodiment of a sleeve according to the invention;

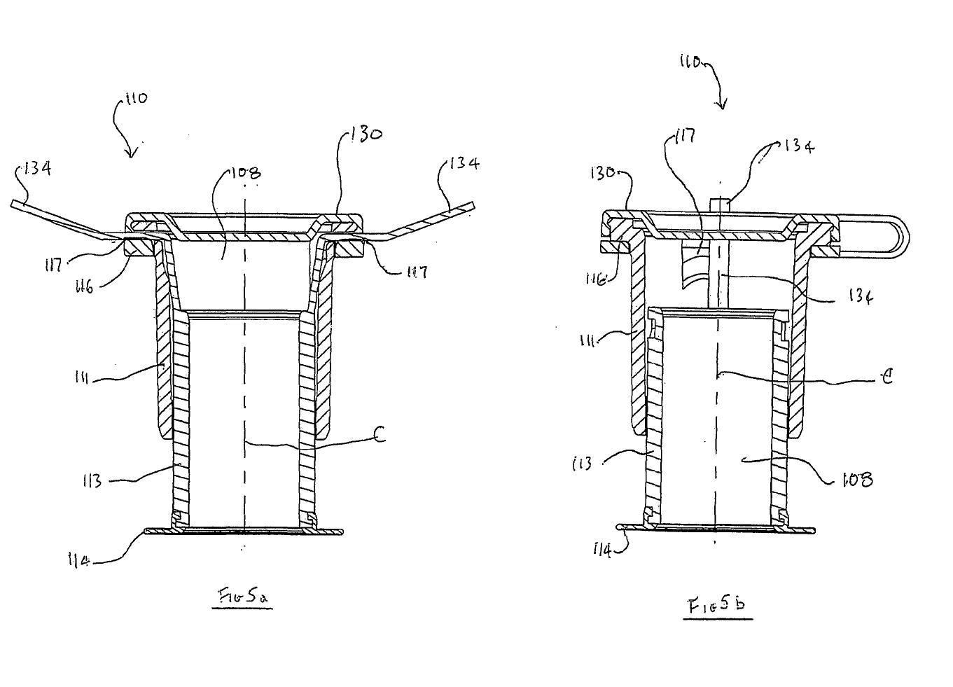

FIGS. 5a, b and 6 show sectional views of the third embodiment;

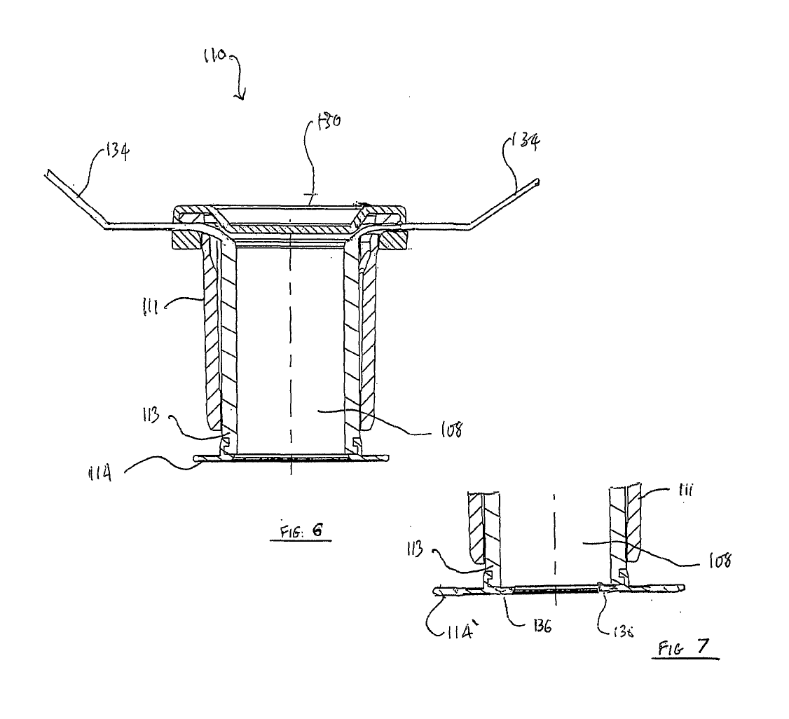

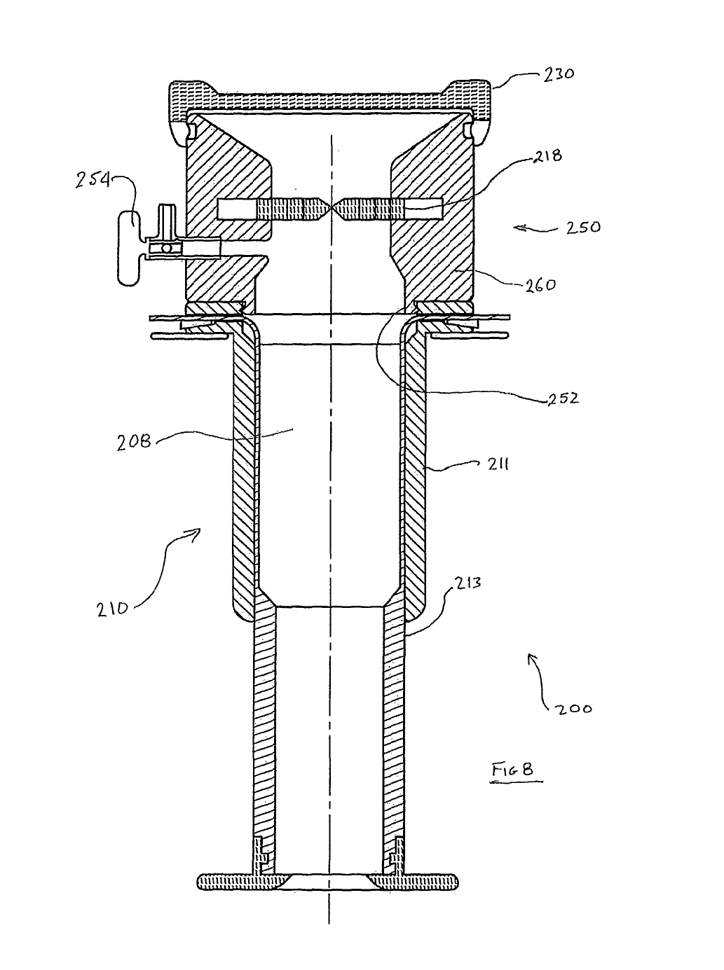

FIG. 7 shows a partial view of a modification to the embodiment shown in FIGS. 5a,b and 6;

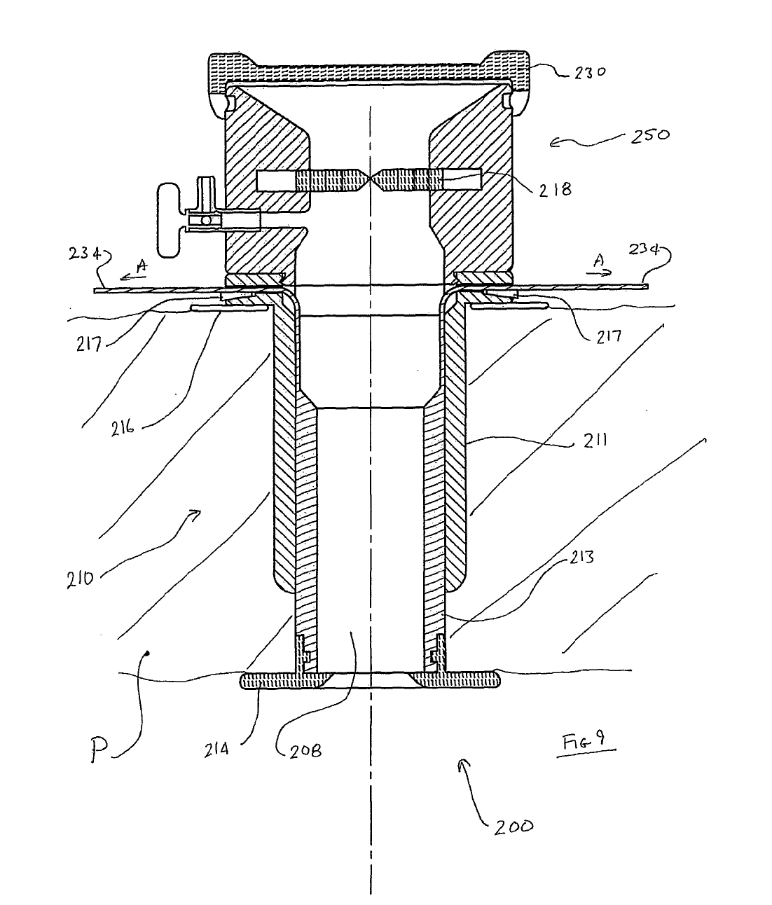

FIGS. 8 to 10 show a fourth embodiment of the invention;

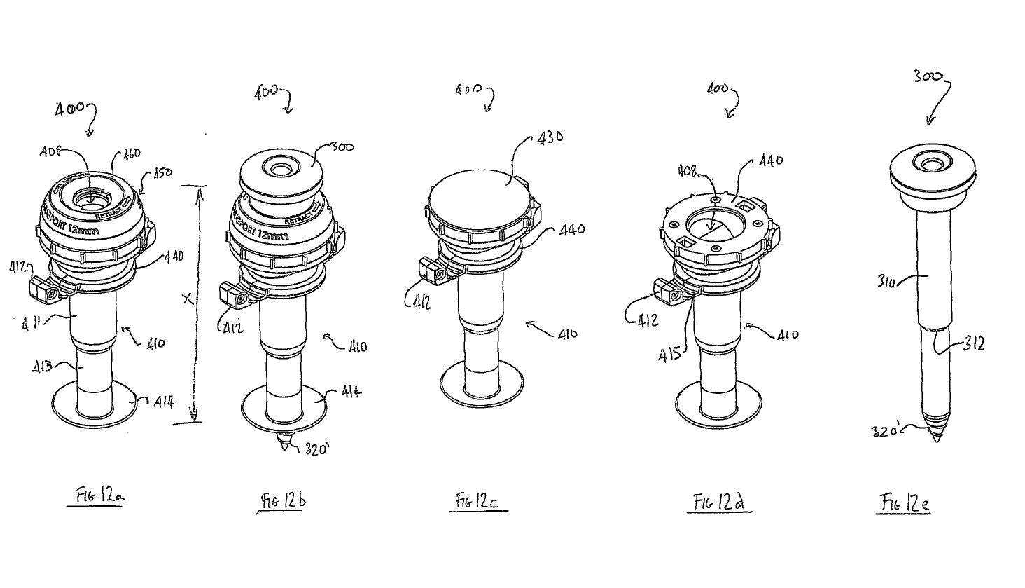

FIG. 11 shows an adaptation of the fourth embodiment;

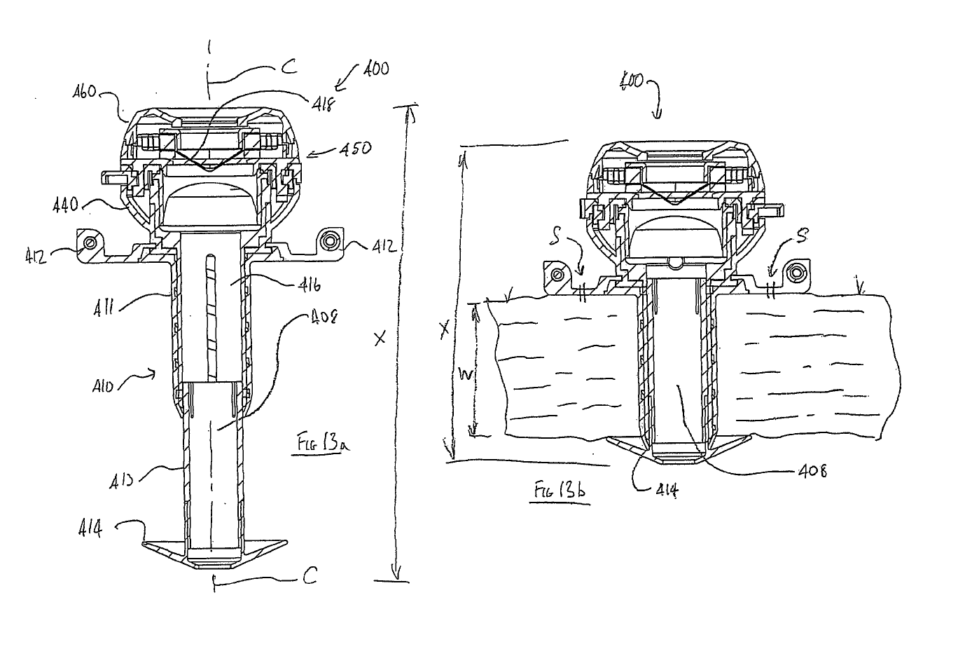

FIGS. 12 to 14 show a further embodiment of the invention; and

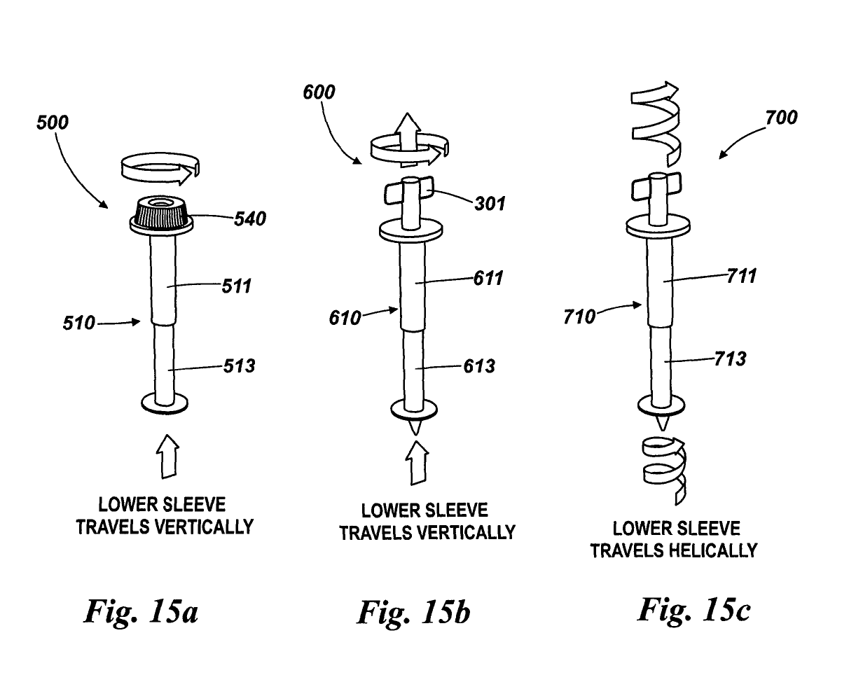

FIGS. 15a to 15c show yet further embodiments of the invention.

Referring to FIG. 1, there is shown generally an arrangement of a sleeve 10 for a laparoscopic access port 50 in use. Initially an incision is made in the patient P and the sleeve 10 is inserted into the incision. The insertion of the sleeve 10 is assisted by the flexible nature of the flanges of the sleeve as described below. Once the sleeve 10 is in position a laparoscopic access port 50 is inserted into the sleeve 10 through a channel 8. The access port 50 is of a generally known construction and so is not described here in detail, although FIG. 1 illustrates a tube 52, which is used for insufflation of a body cavity C using a pressurised gas G supplied by the pipe 52. The access port 50 includes a generally central aperture 54, which is self-sealing and allows the insertion of surgical tools and the like through the aperture 54 and into the body cavity C.

The access port sleeve 10 is shown in more detail in FIGS. 2a, 2b and 2c. FIG. 2a shows the sleeve 10 before it is inserted into the patient P. The sleeve comprises a generally tubular portion 12, an inner flange 14 and an outer flange 16. The sleeve is manufactured from a rigid or semi-rigid plastic such as polythene, and the inner and outer flanges 14 and 16 are manufactured from a flexible elastomer such as silicon rubber.

The inner flange 14 in use sits against the inner wall of a body cavity C (as shown in FIG. 1) and the outer flange 16 lies against the epidermis of a patient P. The sleeve includes a first seal member 18 adjacent the outer flange 16. The purpose of the first seal member 18 is to provide a seal between the sleeve itself and the laparoscopic access port 50 when the access port 50 is inserted within the channel 8 of sleeve 12. In this embodiment the seal member 18 is provided by an inner lip of the outer flange 16. Additionally, the inner flange 14 acts as a further, second, seal member to provide a seal between the sleeve 10 and the inner wall of the body cavity C. The seal members 14 and 18 act to prevent or inhibit the escape of insufflation gases G from within the body C.

Referring additionally to FIG. 2b the tubular portion 12 is shown in more detail. The portion 12 is formed from two tubular pieces 11 and 13 each of which have a series of protrusions which form complementary ratchet formations 15 and 17 which act as a one-way movement mechanism so that the pieces 11 and 13 can be pushed together but not pulled apart. In use, this allows the flanges 14 and 16 to be brought together, but they cannot move apart. Stop members 22 at the ends of the ratchet mechanism 15 and 17 prevent the inner and outer tubular pieces 11 and 13 separating and so this prevents the inner tubular piece 13 from inadvertently falling into the body cavity C.

Referring additionally to FIG. 2c, a section of a circumferential portion of the inner flange 14 is illustrated. The flange has a thickened annular periphery, in this case a ring 20 of circular cross-section. The purpose of the thickened periphery is to help maintain the shape of the flange 14 within the cavity C, and help it spring into shape when it has been collapsed and inserted into the initial incision in the patient P.

A captive plug 30 is illustrated in FIG. 2a which can be inserted into the aperture 8 and sealed against the first seal member 18. The plug 30 includes a membrane 32 which can be punctured so that a drain or the like can be passed through the aperture 8 into the cavity C.

In general the internal diameter of the aperture 8 will be in the order of 7.5 mm to 14 mm to allow the insertion of a laparoscopic access port which in turn is capable of receiving laparoscopic surgical tools of a diameter of 5 mm to 11 mm. Whilst the sizes mentioned above are typical of presently used apparatus, it will be appreciated that other sizes may be employed for example smaller sizes may be used for paediatric surgery.

In use the sleeve 10 is pushed through an incision in the patient P by collapsing the inner flange 14 and forcing the sleeve 12 into the incision. As the inner flange 14 enters the cavity C the flange will resiliently return to its planar shape as illustrated in FIG. 2a. In this position the ratchet mechanism 15 and 17 can be used to draw the inner flange 14 and outer flange 16 together. When the two flanges are drawn together the outer flange 16 will sit neatly on the outside of the patient P generally flush with the patient's skin. The laparoscopic access port 50 can then be repeatedly inserted and removed from the patient into and out of the channel 8, without the unnecessary trauma to the patient P.

When the access port 50 is removed the plug 30 can be inserted into the channel 8 so as to avoid infection entering the cavity C via the channel 8.

A second embodiment of the invention is shown in FIGS. 3a and 3b. Where features of the first and second embodiments are similar or identical then these features have like reference numerals. This sleeve 100 is used in a similar manner to the sleeve 10 described above. There are two flanges 14 and 16 on either side of tubular pieces 11 and 13. Lower tubular piece 13 includes two straps 102 extending upwardly, through flange 16, and through plug 30. The straps are held together by a sprung clasp 104. In use, when the sleeve is inserted into a body cavity or the like, the clasp can be he held with one hand, whilst the upper flange 16 is pushed away from the clasp. Since the straps hold the lower tubular piece 14 in place, then the flanges will be forced together as the flange 16 is pushed.

FIG. 3b shows a one-way ratchet mechanism which is similar to the to ratchet mechanism shown in FIG. 2b. In FIG. 3b only one set of ratchet formations 17 is present, which act with end stops 22 to provide one-way movement, for moving the flanges 14 and 16 together. Since there are no features of the ratchet mechanism within the channel 8 then the channel provides easier passage for access ports and the like, and makes the parts easier to produce.

In use the flanges can be brought together as described above. When it is desired to seal the channel 8, the plug 30 is pushed into the channel 8 and the clasp 104 is moved down relative to the straps 102 to prevent the plug from coming out.

In a refinement, the lower tubular piece 13 is slightly longer than the upper tubular piece 11. This means that the upper tubular piece cannot be inadvertently forced to protrude into a body cavity beyond the flange 14, so reducing the likelihood of damaging internal organs and the like.

A third embodiment of the invention is shown in FIGS. 4 to 7. The general arrangement of parts in the third embodiment is similar to the previously described embodiments. This embodiment is used in the same way as, and has similar dimensions to the previously described embodiments

Referring to FIGS. 4a and 4b a sleeve 110 is pushed into an incision in a patient and left there until it is no longer required, allowing repeated insertions of laparoscopic access ports and the like. The sleeve 110 includes an moulded plastics inner tubular piece 113 and a moulded plastics outer tubular piece 111 which are relatively adjustable by means of a telescoping movement in the direction of arrows A along axis C, as the outer tubular piece 111 slides over the inner tubular piece 113. Insertion of the sleeve 110 into a body cavity is carried out by holding the outer tubular piece 111 and forcing the inner piece 113 into the cavity. Since the action of inserting of the sleeve into a body cavity is likely to require some insertion force, then this could result in the collapsing of the tubular pieces together. However the collapsing is prevented by a mechanism including a bayonet type fitting which includes a channel 115 and is described in more detail below.

The sleeve 110 includes an inner flange 114 and an outer flange 116, which perform sealing functions as described above. The outer tubular piece 111 has a cap 130, which is used for sealing the sleeve when not used for laparoscopic access. The cap 130 has a tether 132.

FIGS. 5a and 5b show sections of the tube when they are partially collapsed. Such a partial collapsed state may provide the correct dimension between the inner and outer flanges, for example if the patient is overweight.

Access channel 108 for accepting a laparoscopic access port can be readily seen formed within inner diameters of the two tubular pieces 111 and 113. Two flexible draw tabs 134 can be seen. These are integrally moulded with the inner tubular piece 113 and extend through openings 117 in the outer flange 116. The tabs 134 can be gripped and pulled through the openings 117 from the outside to hold the inner piece 113, while the outer tubular piece 111 is pushed toward the inner piece 113. This action brings the two tubular pieces together. The openings 117 include a gripping mechanism for one-way movement of the tabs 134 therethrough so that the two tubular pieces are held together by tension of the tabs and cannot come apart once they have been forced together.

FIG. 6 illustrates the third embodiment wherein the two tubular pieces 111 and 113 have been collapsed fully because the draw tabs 134 have been pulled to their fullest extent. This arrangement would be suitable for patients having a thinner cavity wall.

A modification is shown in FIG. 7, wherein the flange 114′ is radially extended compared to the flange 114 shown in FIGS. 4a to 5c. This extended flange is approximately 1.5 times the diameter of the bore 108. Also, the flange 114′ has an inner lip 136 which seals against a port 50 or the like when the port is inserted into the bore 108. This lip is preferably in the position shown, but could be positioned anywhere in the bore 108.

Inner flange 114 is resiliently fitted to the inner end of the moulded tube and the draw tabs 134 are integrally formed on the outer end of this tube. The tube wall includes the channel 115 which has a majority of its length extending parallel to the axis C. At the outer end of the tube 119 the channel extends obliquely to the axis. A similar mirror-image channel (not shown) is provided on the opposite side of the tube.

The outer tubular piece 111 and cap 130 in more detail. The outer tube 111 includes a pair of studs 118, slideable in the channels 115 mentioned above. The studs and channels form a bayonet fitting which restricts the coming together of the two tubular pieces 111 and 113 when the studs are located in the outer oblique end 119 of the channel 115. When the outer tubular piece 111 is then rotated about axis C relative to the inner tubular piece 113, the studs can be moved into a position in the channel, which allows the tubular pieces to come together. It will be noted that the openings 117 are wider than the tabs 134 to allow said relative rotation of the tubular pieces.

The embodiments described above refer to a sleeve which can accommodate a conventional access port. However, in an alternative design a sleeve having a similar arrangement to the sleeves described above may form part of a surgical access port.

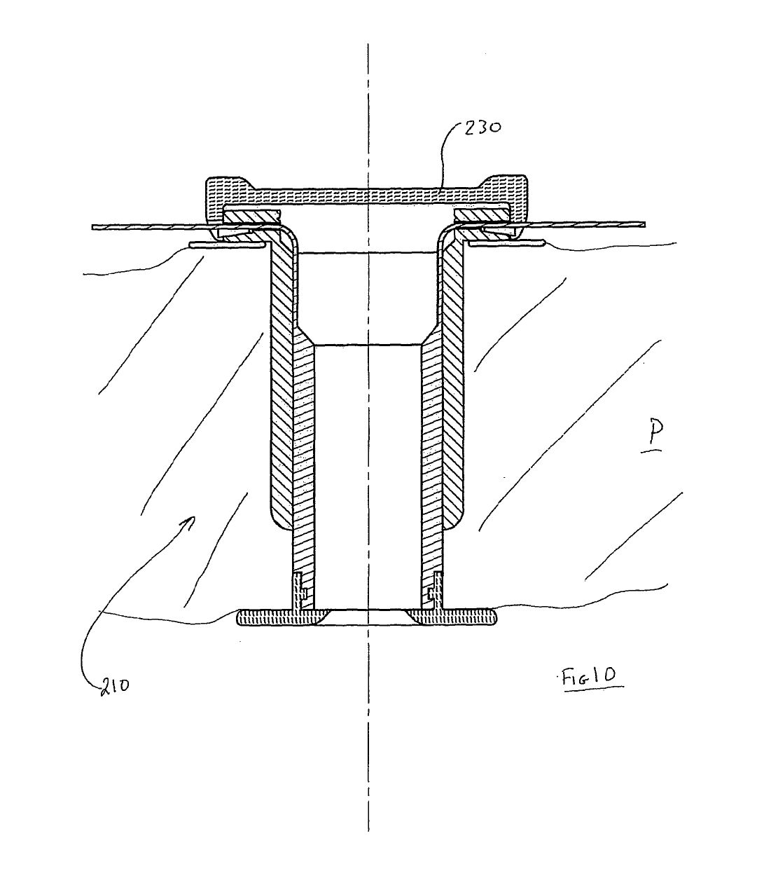

Such a combined port and sleeve 200 is illustrated in the sectional views of the fourth embodiment shown FIGS. 8, 9 & 10. In this embodiment sleeve 210 is of similar construction to the sleeves 10, 100, and 110 mentioned above, and although not explicitly illustrated, it is envisaged that the sleeve 210 may have incorporated, the same or similar attributes as the sleeves 10, 100 and 110. Parts which are similar to the parts of sleeves 10, 100 and 110 have like numbering.

Referring in particular to FIG. 8, sleeve 210 is removably attached to the head of an access port 250 by means of a shoulder 252 on formed integrally with a body portion 260 of the port head 250, which is a snap fit into an aperture 208 of the sleeve 210. Access port head 250 includes a cap 230, a spiral seal 218 and a gas supply tap 254.

In use, as illustrated in FIG. 9, the sleeve 210 is inserted into the incision in a patient P and pushed through the body tissues. The sleeve 210 is telescopic, because the outer piece 211 of the sleeve 210 and the inner piece 213 of the sleeve may slide relative to each other. However, when inserted into the patient, it is envisaged that the inner and outer pieces will be restrained against their relative movement, for example by the use of a bayonet fitting of the type described above and illustrated in FIG. 4a.

Following insertion, the inner and outer pieces are relatively rotated to remove the influence of the bayonet restraint. Draw tabs 217 are pulled in the direction of arrow A and as a result, the inner piece 213 is drawn toward an outer piece 211 of the sleeve 210. A one way movement mechanism 217 stops the tabs from sliding backwards. As described previously, flanges 214 and 216 on the inner and outer parts, come closer together to minimise the leakage of insufflation gases. The flanges are formed from elastomeric material.

At this stage, cap 230 can be removed and an instrument (not shown) can be pushed through the seal 218 into the aperture 208 and on, into the patient P.

After surgery, the instrument is removed and the port arrangement 200 can be removed. In this embodiment, the head 250 can be removed from the sleeve 210, whilst the sleeve remains in the patient. Thus the sleeve can remain in the patient for further access to the surgical site in subsequent surgical operations, or for observations, or drainage, or suchlike, in a similar manner to the sleeves described above.

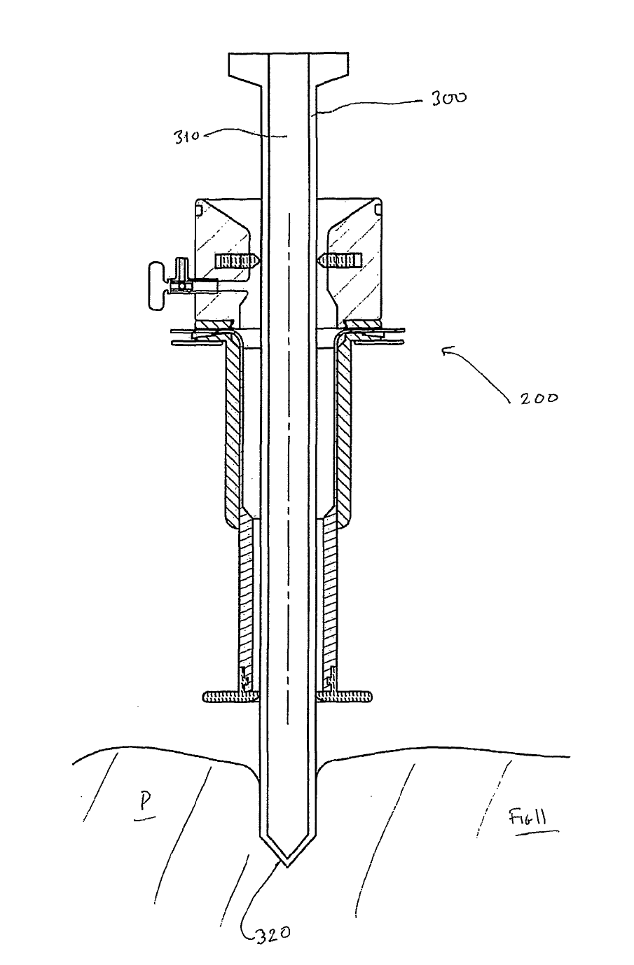

FIG. 10 shows the sleeve 210 with the access port 250 removed, for allowing the sleeve to remain in the patient if desired. In this case the cap 230 has been fitted over the sleeve to prevent the ingress of contamination. It should be noted that, in this embodiment, it is not essential that the head 250 be removable from the sleeve 210, however when the head is removable, then the patient need not endure discomfort if the sleeve is left in place.

In FIG. 11, the access port arrangement 200 includes a pointed introduction tool 300 for aiding the introduction of the sleeve 210 into the patient P. The tool 300, has a hollow interior including a bore 310 which in use can be used to insert a camera to aid the guiding the tool 300 during insertion by means of inserting the camera to the pointed end 320 of the tool 300.

Referring to FIGS. 12a to 12e a further laparoscopic access port 400 is illustrated in different configurations. In FIG. 12a, the port is shown having two main parts—an access head 450 which remains external to a patient, and provides access for laparoscopic surgical instruments through an access bore 408, and a sleeve 410 which is insertable into an incision in a patient and thus is generally internally disposed in use.

The generally tubular sleeve 410 has two main parts—a shank 411 generally rotatably attached to the head 450, and a moveable flanged piece 413, which is moveable relative to shank 411 to adjust the overall length X (including the head) of the port 400. A mechanism is described below for causing said movement.

The head 450 is divided into two parts—a lower part 440 which is rotatably attached to the shank 411 and a removable part 460, which can be detached, as shown in FIG. 12d, for the reasons mentioned above relating to patient safety and comfort. As shown in FIG. 12c, a cap 430 can be fitted over the lower port part 440.

As illustrated in FIGS. 12b and 12e, the port 400 can be inserted into a patient using the introduction tool 300 described above, again fitted temporarily within bore 408. The introduction tool shown in FIGS. 12b and 12e includes an auger 320′ which aids insertion of the port 400. The introduction tool includes a shoulder 312 which sits on the externally facing end of the flanged piece 413 to inhibit that flange piece from collapsing into the shank during insertion, as well as minimising the stress exerted on the length adjustment mechanism described above.

Once inserted a flange 414 helps to hold the port in place as described above and provides a radially inwardly facing seal around bore 408 to aid prevention of the escape of insufflation gases when a laparoscopic tool is being used in the bore 408. Sutures stitched into the skin of a patient, can be attached to ears 412 to aid the securing of the port 400 in place on/in the patient's body. The ears include tapering slots 415 for capturing and securing the sutures in place.

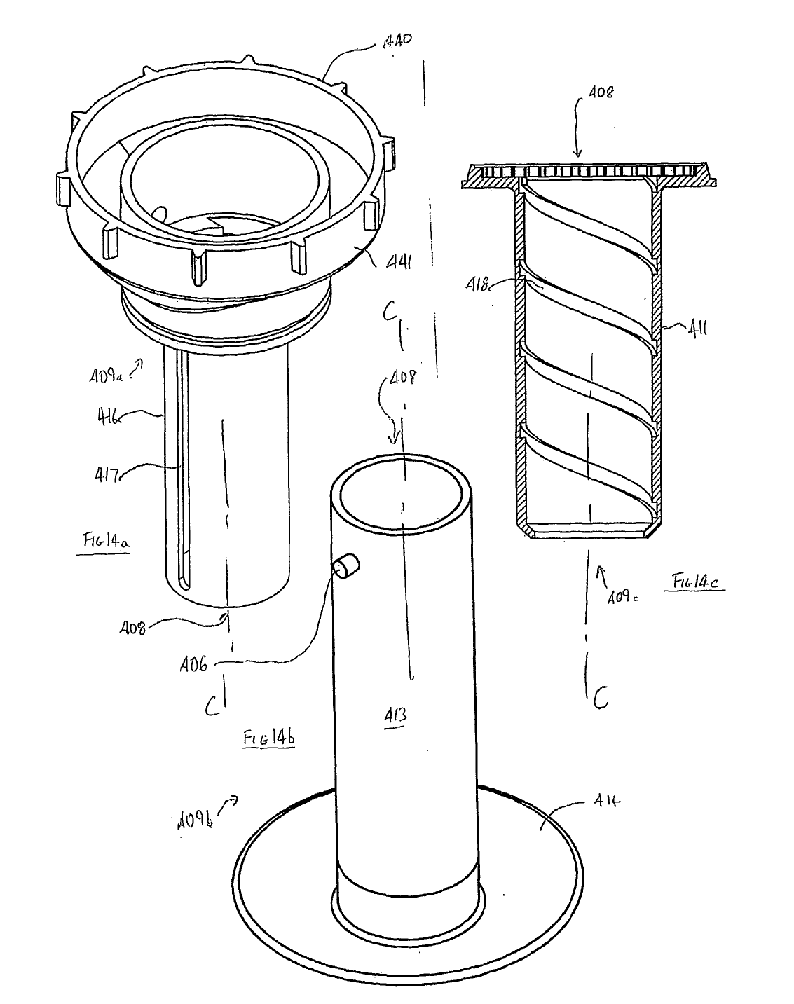

FIG. 13a is a longitudinal section through FIG. 12a and FIG. 13b is a section similar to the section shown in FIG. 13a but with the flange 414 in a retracted position and shown in use. Referring to these two Figures, the head 450 includes a seal member 418 which is displaced when a laparoscopic instrument (not shown) is inserted into the head 450 generally along an axis C, but otherwise inhibits the passage of gases. In use the flanged piece 413 is telescoped into the shank 411 so the overall length X is reduced to fit the abdominal wall thickness W of the patient and sutures S are used to hold the port in place and the flange 414 seals the port against the patient's cavity wall.

Referring to FIGS. 14a,b and c, an exploded view of the mechanism 409a, 409b and 409c (collectively 409) for moving the flanged piece 413 is shown. The mechanism includes an internal thread 418 formed in the inside of the shank 411 and diametrically opposed detents 406 formed on the outside of the flanged piece, only one of which is visible. The detents 406 can ride in the thread 418 such that relative rotation of the shank 411 and flanged piece 413 causes the flanged piece to move axially along axis C. Said relative rotation is caused by an intermediate tubular piece 416 attached to the lower port part 440. This intermediate part in use lies radially between the shank 411 and the flanged piece 413 and the detent 406 extends captively through slots 417 in the intermediate piece (only one of which is visible), and into the thread 418. Rotation of a collar 441 on the port part 440 causes said rotation of the flanged piece 413 driven by the detent 406 and thus said axial movement of the flanged piece 413 following the helix of the thread 418. It is envisaged that more then one detent may be formed on flanged piece 413, and a complementary number of slots 417 can be provided also.

Various alternatives to the above embodiments are described below with reference to FIGS. 15a, b and c which each show modifications of the previously described embodiment. Referring to FIG. 15a a three-tube access port arrangement 500 is illustrated, in which a sleeve 510 has three tubes one inside the other, similar to the tubular arrangement mentioned above. However, in this case a thread is formed on the intermediate tube, which is rotatable externally of the patient by rotation of collar 540, as described above. The flanged piece 513 can follow the thread and is prevented from rotating with the intermediate piece by detents extending from a shank portion 511, which extend into slots in the flanged piece 513. Thus the flanged piece moves purely axially rather than the helical path of the flanged piece 413. Thus a mechanism is shown which again allows adjustment of the overall length of the access port externally of the patient.

Referring to FIG. 15b a further access port arrangement 600 is illustrated, including a sleeve 610, and wherein, an introducer tool 301, is temporarily held in position in a flanged piece 613. The introducer acts as a handle to pull or push the flanged piece 613 relative to a shank portion 611, in the manner of a syringe plunger. Thus again a mechanism is shown which allows adjustment of the overall length of the access port externally of the patient using two tubular pieces 611 and 613.

Referring to FIG. 15c a further access port arrangement 700 is illustrated, wherein introducer 301 is used to rotate the complementarily threaded shank 711 and flanged piece 713, of sleeve 710, to provide a further mechanism which allows helical adjustment of the overall length of the access port externally of the patient, again using two tubular pieces 711 and 713.

It will be readily apparent to the skilled addressee that further modifications, alterations and additions to the embodiments described above are possible. For example, suitable alternatives to the materials used could be employed. Thus, the flexible silicon elastomer described could be replaced by other suitable biocompatible material i.e. material which is safe for use within a patient's body cavity. The sleeves 10, 100, 110, 210, 410, 510, 610 and 710 could be manufactured from other plastics materials, or metals or ceramic materials. It is possible that the sleeves could be made completely or partially from biodegradable material so that disposal is made less expensive, or from material which can be cleaned and reused. Plastics materials described could include a bactericide to reduce the likelihood of infection. The inner and outer flanges are illustrated as being generally planar and parallel, although it is envisaged that for some applications the shape of the flanges could be altered. For example, the outer flange could be shaped to fit a particular curved body part and thus avoid protruding too far above the skin of a patient. A circular channel 8, 108, 208, 408 and flanges 14, 114, 214, 414 and 16, 116, 216 are preferred but it will be appreciated that other shapes, for example hexagonal, could be used. The terms tube and tubular are intended to embrace such non-circular shapes.

1. A laparoscopic access port having: a head portion for use generally externally of a patient and for providing a seal around a laparoscopic tool when said tool is inserted into the head; and a sleeve portion connected to the head and for accommodating the tool, the sleeve extending along an axis and being adapted for inserting generally into the body of a patient in the direction of the axis; said access port being adjustable in overall length in the direction of the axis, the sleeve portion comprising a shank attached or attachable to the head and a flanged piece moveable relative to the shank by means of a mechanism for causing the overall length of the port to adjust, the mechanism being operable externally of the patient at or adjacent the head.

2. A laparoscopic access port as claimed in claim 1, wherein the head, or part of the head, and sleeve are separable.

3. A laparoscopic access port as claimed in claim 1, wherein said sleeve is generally tubular, including a first tubular piece forming the shank and a second tubular piece forming the flanged piece, one of said tubular pieces being relatively moveable within the other to adjust the overall length of the port.

4. A laparoscopic access port as claimed in claim 3, wherein either the first or second tubular piece has a thread and the other of the first or second tubular pieces has a complementary thread or thread-following formation such that the movement of the second tubular piece causes said overall length adjustment.

5. A laparoscopic access port as claimed in claim 4, wherein the rotation of the second tubular piece is caused by a manually rotatable third tubular piece forming part of the mechanism and extending from the head such that the third tubular piece can be rotated externally of the patient in use, by rotation of the head or part of the head.

6. A laparoscopic access port as claimed in claim 4, wherein the first and second tubular piece do not rotate relative to each other in use.

7. A laparoscopic access port as claimed in claim 5, wherein said third tubular piece extends at least partially within the second tubular piece, which in turn is at least partially within the first tubular piece.

8. A laparoscopic access port as claimed in claim 7, wherein the third tubular piece includes at least one axially extending slot for accepting a detent formed on or in the second tubular piece for allowing the detent of second tubular piece to move axially in the slot under the influence of the thread in the first tubular piece.

9. A laparoscopic access port as claimed in claim 1, where the flanged piece includes a distal end at which is formed a flange, optionally being formed from flexible material.

10. A laparoscopic access port as claimed in claim 1, wherein the mechanism includes an indexing mechanism providing audible and/or tactile indication of movement of the second tubular piece.

11. A laparoscopic access port as claimed claim 1, wherein the mechanism includes at least one flexible draw tab attached to the flanged piece for holding the flange in place while the shank is moved relative to flanged piece.

12. A laparoscopic access port as claimed claim 1, wherein the mechanism includes an introduction tool insertable into the flanged piece or second tubular piece, and extending such that the introducing tool can be manipulated externally of the patient to move the flanged piece relative to the head.

13. A laparoscopic access port as claimed in claim 1, wherein the mechanism includes an introduction tool insertable into the flanged piece, said tool including a shoulder for engagement with the flanged piece for supporting said flanged piece during insertion of the port into a patient and maintaining the port in an extended position.

14. A laparoscopic access port as claimed in claim 1, wherein the port may further include a plug or cap to substantially seal the port after the head of the access port is removed or partially removed.

15. A laparoscopic access port as claimed in claim 1, wherein the head includes a pierce-able septum.

16. A laparoscopic access port as claimed in claim 1, further including at least one ear at or adjacent the head for allowing securing of the head by means of suture, said ears including V shaped open slots for capturing said suture.

17. A sleeve arrangement for accepting laparoscopic equipment, comprising: a sleeve portion extending along an axis and being adapted for inserting generally into the body of a patient in the direction of the axis;
said sleeve portion being adjustable in overall length in the direction of the axis, the sleeve portion comprising a shank attached or attachable to an external portion of the sleeve arrangement and a flanged piece moveable relative to the shank by means of a mechanism for causing the overall length of the sleeve to adjust, the mechanism being operable externally of the patient at or adjacent the external portion.
